(19) United States
(12) Patent Application Publication
(10) Pub. No.: US 2026/0191347 A1
(43) Pub. Date: Jul. 9, 2026

(54) STRESS OFFLOADING PILLOW

(71) Applicant: BIOCONIX PTY LTD, Wentworth Falls, New South Wales (AU)

(72) Inventors: Melissa Knothe Tate, New South Wales (AU); Ulf Knothe, New South Wales (AU)

(21) Appl. No.: 19/132,030

(22) PCT Filed: Nov. 22, 2023
(86) PCT No.: PCT/IB2023/061808

§ 371 Date: May 22, 2025

Related U.S. Application Data
(60) Provisional application No. 63/384,705, filed on Nov. 22, 2022.

(51) Int. Cl. A47G 9/10

(52) U.S. Cl. CPC A47G 9/1036; A47G 2400/10

(57) ABSTRACT

A pillow for offloading stress on a body part in need thereof includes a stress dissipating support with a void extending there through configured to offload stress from the support and a cover material extending over the support and void configured to offload stress to body part of interest, while also minimizing chafing or friction or stress concentrations at surfaces where the pillow meets skin of the body part.

### RELATED APPLICATION

This application claims priority from U.S. Provisional Application No. 63/384,705, filed Nov. 22, 2022, the subject matter of which is incorporated herein by reference in its entirety.

### BACKGROUND

Various arrangements have been proposed in the past, for providing cushions and pillows, as a means to rest a person's head and other parts of their body. They are available in a wide variety of designs, materials, shapes, and sizes depending upon their intended use. From a conventional rectangular pillow used in bed, primarily for a person's head; to decorative cushions and throw pillows, for use on chairs, sofas, and recliners; to a wide variety of cushions and pillows made for special applications, such as body, bolster, donut, lumbar, neck, orthopedic, sleeve, sofa, and travel. By definition, a cushion or pillow, is a cloth case stuffed with something soft, such as down, feathers, or foam.

A body of scientific literature cites the benefits of sleeping with a pillow between, e.g., osteoarthritic knees, for mitigation of pain. Such pain often intensifies during sleep and/or rest periods, when the knees are unsupported at different angles to the hip and rub uncomfortably against one another. The resulting reduction in sleep and rest feeds into a cycle of pain when loading painful joints and body parts after rest, i.e., during activity. Similarly, individuals who sleep on their side and/or stomach place stresses and strains on the skin and underlying structures of the face, including the teeth.

### SUMMARY

Embodiments described here relate to a novel stress offloading pillows that can be used stress offloading of, for example, surgical sites, e.g., after mastectomy with/without reconstruction, infections, to prevent decubitus ulcers and to facilitate healing of already present decubitus ulcers, and hemorrhoids. Most pillows are designed to support body parts, including the head, neck, knees, and back, through material and or structural support. The stress offloading pillow described herein is designed to offload (protect) body parts from stress (tension or pressure or torsion or shear), using a combination of voids (cut-outs), geometries and structural compliance, which enables simultaneous distribution and or dissipation of stress at interfaces with the body. The offloading of affected body parts results in reduction of internal residual stress as well as externally applied stresses during rest. This simultaneously reduces stress and strain (e.g., folding) of the skin between the void and the body part as well as resetting of the internal stress state of the body part, which is designed to improve sleep, reduce pain, promote healing, improve vascularization and reduce decubitus ulcers and infections, e.g., after injury and surgical repair.

In some embodiments, the stress offloading pillow includes a stress dissipating support with a void extending there through configured to offload stress from the support and a cover material extending over the support and void configured to offload stress to body part of interest, while also minimizing chafing or friction or stress concentrations at surfaces where the pillow meets skin of the body part.

In some embodiments, the stress includes at least one of tension, pressure, torsion, or shear on the body part.

In other embodiments, the support is configured with respect to the geometry and compliance of the body part to be offloaded.

In some embodiments, the pillow is self-stabilizing and free-of straps for maintenance of single position to remain in place.

In some embodiments, the pillow further includes a filling configured to be cooled or warmed for application heat or cold as desired.

In other embodiments, the pillow further includes at least one an electromechanical stimulator or sensor for delivery of infrared and/or other wavelength energy.

In some embodiments, the stress dissipating support includes round edges to distribute or dissipate stresses.

In some embodiments, the cover material can have a coefficient of friction substantially similar to the coefficient of friction of skin of the body part.

The benefit of offloading pillows extends to clinical contexts for the prevention of pressure sores (decubitus ulcers) and/or offloading of body parts and/or sites to facilitate healing, e.g. after surgery, therapy, etc.

### BRIEF DESCRIPTION OF THE DRAWINGS

FIG. 1 illustrates a schematic drawing of a knee off-loading pillow supporting knees of subject.

FIG. 2 illustrates a schematic top plan view of a knee off-loading pillow in accordance with an embodiment.

FIG. 3 illustrates a schematic top plan line drawing of the knee off-loading pillow in accordance with an embodiment.

FIG. 4 illustrates a schematic side plan line drawing of the knee off-loading pill in accordance with an embodiment.

FIG. 5 illustrate a schematic top plan view of a face off-loading pill in accordance with an embodiment.

FIG. 6. Illustrates a schematic top plan line drawing of face off-loading pillow in accordance with an embodiment.

### DETAILED DESCRIPTION

Embodiments described here relate to novel stress offloading pillows that can be used stress offloading of, for example, surgical sites, e.g., after mastectomy with/without reconstruction, infections, to prevent decubitus ulcers, and to facilitate healing of already present decubitus ulcers, and hemorrhoids.

In some embodiments, the stress offloading pill can include a knee offloading pillow. The knee offloading pillow can be used for sleep and rest aid to reduce pain, to promote healing, to reduce risk of infection, and to facilitate movement throughout life.

In other embodiments, the stress offloading pill can include a face offloading pillow. The face offloading pillow can be designed for offloading of the face during sleep on the side or stomach. Sleeping on the side or stomach applies stress and strain to the face. Over time, these stresses and strains can displace teeth (change bite patterns) as well as cause permanent folds in the skin. The pillow can be manufactured with a large cutout to accommodate the face and unimpeded breathing via the nose while the support of the head is maintained.

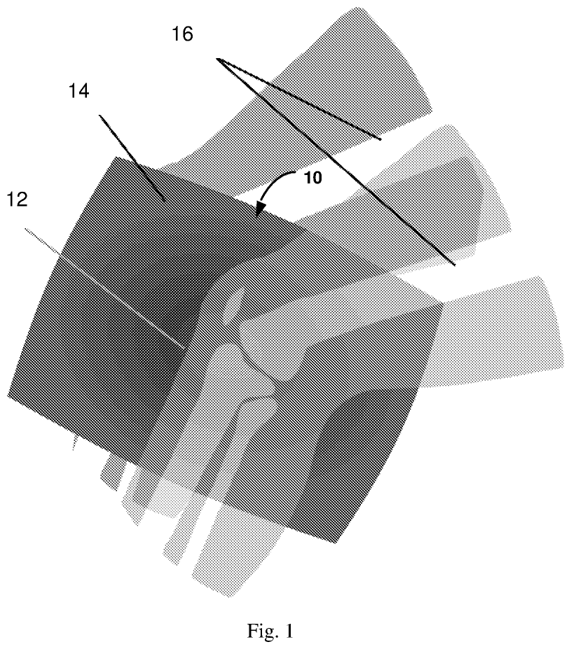

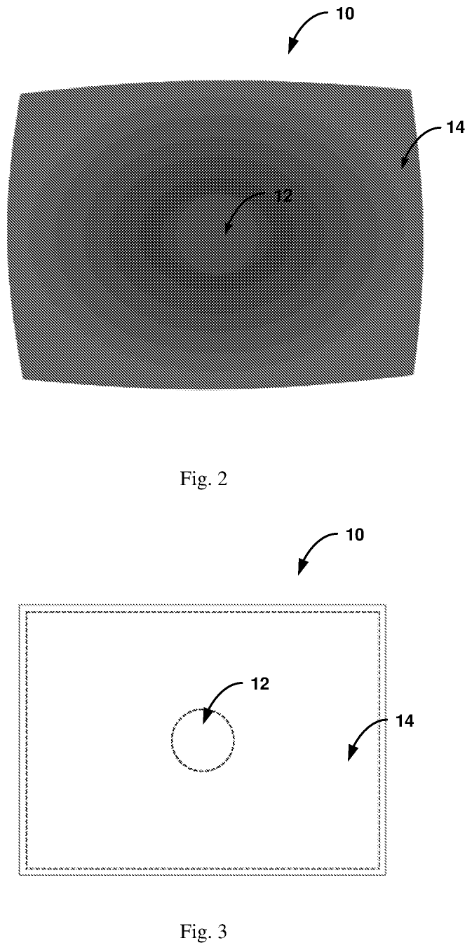

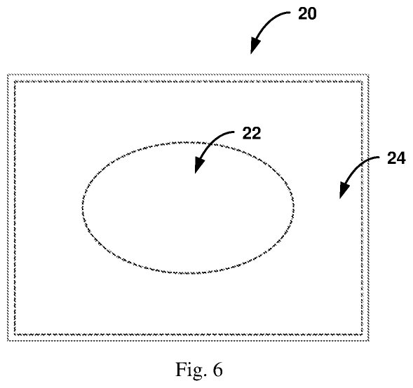

FIG. 1 illustrates an example of a stress offloading pillow 10, that is characterized by a void (cutout) 12 which off loads, e.g., the knees 16, when the user sleeps on their side with the knees together. The pillow 10 can be created with different external dimensions and shapes as well as interior, off loading voids (cut outs) 12. For example, as illustrated in FIGS. 5 and 6, to protect the face and teeth during side and stomach sleeping, the offloading pillow 20 supports the head and neck while offloading structures of the face and teeth. The external dimensions and shape contribute to stability of the pillow 10 or 20 throughout the sleep cycle; e.g., in contrast to currently available knee pillows that move and/or get displaced when the user turns in their sleep. The design enables easy adjustment, i.e., can be turned to increase or decrease the area of exposure to the void and/or interface between the pillow and skin. The pillow self-stabilizes and requires neither straps nor maintenance of a single sleep position to remain in place. The pillow geometry and mechanical properties can be adjusted to optimize for specific body parts, from off loading joints to the face or, in a more clinical context, surgical sites.

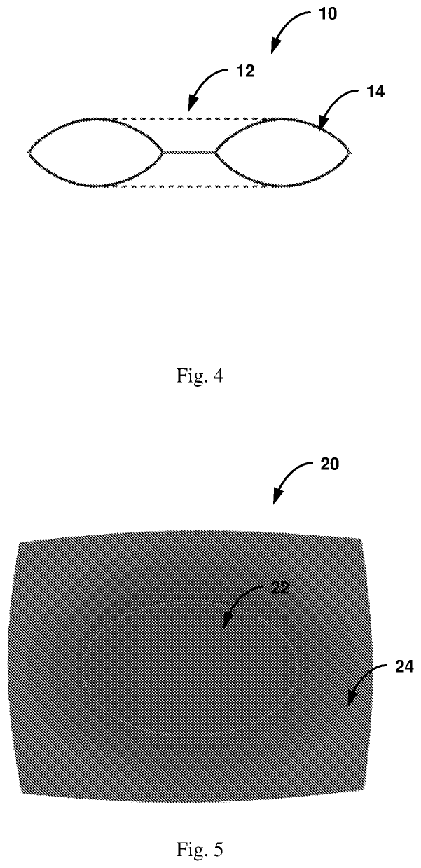

The distribution of stress and strain at the interface between a human body part and environmental support, e.g., a mattress or pillow during sleep or rest, depends on the geometry and compliance of the support. Hard supports with edges concentrate stresses; they can cause discomfort and pain, as well as disrupt vascular and lymphatic flow, which itself increases risk of decubitus ulcers and infection. Soft supports 14, 24 with round edges distribute or dissipate stresses. Placement of voids 12 and 22 (cutouts, FIG. 1, FIG. 3, FIG. 4, and FIG. 6), together with engineered compliant and stress-dissipating supports 14 and 24 (FIG. 1, FIG. 3, FIG. 4, and FIG. 6) with, for example, rounded edges, can be designed to offload specific body parts from stress to an even greater extent. The core material of the support is selected with respect to the geometry and compliance of the body part to be offloaded. A covering material that extends over the void is selected to either mimic skin (e.g., hypoallergenic microfiber or ultra-suede, for cushioning between the knees or other joints) or to minimize friction (e.g., raw silk, for cushioning and offloading the face during side or stomach sleeping).

In some embodiments, the compliance of the pillow is designed, together with the cover material to offload stress to the joint or face or body part of interest, while also minimizing chafing or friction or stress concentrations at surfaces where the pillow meets the skin.

The dimension and geometry of the pillow can be adapted to best address specific body parts as well as to fit specific individuals, e.g., using digital acquisition and rapid manufacturing methods. For example, the pillow design can be adapted to match patient size, specific joint geometries, specific sites of injury and/or therapy and/or surgical repair to facilitate healing.

In addition to different internal and external shapes and sizes, the pillow can be configured with different fillings, such as wheat grain, beads of different compliance, air or gel, that can be e.g., cooled in the freezer or warmed in the microwave for application of heat and cold as desired.

The interior can alternatively be equipped with electromechanical stimulators such as massage rings or other types of sensors for delivery of infrared and/or other wavelength energy.

It is anticipated that the cushions will find use to increase the quality of life with aging as well as in therapeutic and rehabilitations contexts after injury, surgery, and/or as a preventative measure.

In some embodiments, the stress offloading pillow acts like a joint between body parts (knees) or between a body part and the mattress or between a body part and a static seat or stop during rest. In this context, the stress off-loading pillow enables the neck or knees or body part in the central portion to relax to its lowest energy state by maximally dissipating the stress in the connecting body parts, and minimizing stress concentrations between the pillow and the body part, resulting in achievement of a position of least stress and strain, and consequentially a position of maximal relaxation and repose.

In some embodiments, the stress offloading pillow further reduces friction and associated shear stress between body parts (e.g., knees) or between body part and stationary surface, through choice of a coefficient of friction best matching that of the relevant body parts. Textile fiber coefficients of friction (m) range from 0.1 to 0.8.

For example, the palm of the hand has the highest coefficient of friction for skin on body parts as 0.62+/−0.22, and is most closely matched by the material silicone (0.61+/−0.21). The coefficient of friction for the medial surface of the knees when they rub together can be approximated by the anterior and posterior surface of the leg, which have been measured as 0.40+/−0.10 and 0.40+/−0.09, respectively, i.e., nearly identical. For comparison, the static coefficient of friction of bamboo fiber-based textiles is reported as 0.30-0.41, and subjective perception of bamboo fabric for knee offloading pillow covering as superior to slipperier (lower coefficient of friction) textiles.

In contrast, the coefficient of friction of the forehead and face is lower than that of the dorsal hand, which is 0.47+/−0.12. Silk textiles with parallel fibers have a coefficient of friction of 0.53. Silk pillowcases have found broad use for hair and face effects, including putative prevention of wrinkles and reduction of electrostatic interactions with hair.

Advantageously, the compliance of the pillow is designed, together with the cover material to offload stress to the joint or face or body part of interest, while also minimizing chafing or friction or stress concentrations at surfaces where the pillow meets the skin. The dimension and geometry of the pillow can be adapted to best address specific body parts as well as to fit specific individuals, e.g., using digital acquisition and rapid manufacturing methods.

It is anticipated that the pillows will find use to increase the quality of life with aging as well as in therapeutic and rehabilitations contexts after injury, surgery, and/or as a preventative measure. Offloading the joint enables reset of internal residual stress (force balance) during periods of rest.

Better sleep with more options for comfortable positioning and alleviating pain during sleep and rest, e.g., decreased torsion pain in the lower back by aligning upper legs in parallel (hip to knee in parallel between two legs) and an associated reduction in intake of pain medications.

Advantageously, the pillow is easily positioned and adjustable to optimize the area offloaded (interfacing with void or cut out) and/or supported by the pillow structure (interfacing with pillow material structure). The pillow has no need for straps or ties as it is self-stabilizing.

While this invention has been particularly shown and described with references to preferred embodiments thereof, it will be understood by those skilled in the art that various changes in form and details may be made therein without departing from the scope of the invention encompassed by the appended claims. All patents, publications and references cited in the foregoing specification are herein incorporated by reference in their entirety.

Having described the invention, the following is claimed.

1. A pillow for offloading stress on a body part in need thereof comprising:
a stress dissipating support with a void extending there through configured to offload stress from the support and a cover material extending over the support and void configured to offload stress to body part of interest, while also minimizing chafing or friction or stress concentrations at surfaces where the pillow meets skin of the body part.

2. The pillow of claim 1, wherein the stress comprises at least one of tension, pressure, torsion, or shear on the body part.

3. The pillow of claim 1, wherein the support is configured with respect to the geometry and compliance of the body part to be offloaded.

4. The pillow of claim 1, being self-stabilizing and free-of straps for maintenance of single position to remain in place.

5. The pillow of claim 1, further comprising a filling configured to be cooled or warmed for application heat or cold as desired.

6. The pillow of claim 1, further comprising at least one an electromechanical stimulator or sensor for delivery of infrared and/or other wavelength energy.

7. The pillow of claim 1, wherein the cover material has a coefficient of friction substantially similar to the coefficient of friction of skin of the body part.

8. The pillow of claim 1, wherein the stress dissipating support includes round edges to distribute or dissipate stresses.
